(19) United States
(12) Patent Application Publication
(10) Pub. No.: US 2008/0027269 A1
(43) Pub. Date: Jan. 31, 2008

(54) Methods of using pericardial inserts

(76) Inventor: Michael Gertner, Menlo Park, CA (US)

Correspondence Address: SHAYGLENN LLP, 2755 CAMPUS DRIVE, SUITE 210, SAN MATEO, CA, 94403, US

(21) Appl. No.: 11/731,529

(22) Filed: Mar. 30, 2007

Related U.S. Application Data
(60) Provisional application No. 60/744,199, filed on Apr. 4, 2006.
(60) Provisional application No. 60/868,350, filed on Dec. 3, 2006.
(60) Provisional application No. 60/869,556, filed on Dec. 11, 2006.

(51) Int. Cl. A61M 1/12

(52) U.S. Cl. 600/16

(57) ABSTRACT

Devices, systems and methods are provided which are capable of applying pressure and constraint to the heart and use the pericardium to assist in the application of the pressure and force to the heart.

### CROSS REFERENCE TO RELATED APPLICATIONS

This application claims priority under 35 U.S.C. §119 to: U.S. application Ser. No. 60/744,199 of Michael Gertner, entitled “DEVICES AND METHODS TO OPTIMIZE CARDIAC FUNCTION” and filed on Apr. 4, 2006; U.S. application Ser. No. 60/868,350 of Michael Gertner, entitled “PERICARDIAL INSERT” and filed on Dec. 3, 2006; and U.S. application Ser. No. 60/869,556 of Michael Gertner, entitled “PERICARDIAL INSERT” and filed on Dec. 11, 2006, the disclosures of which are incorporated herein by reference.

This application is related to the following applications: U.S. patent application Ser. No. 10/974,248 by Michael Gertner, M. D. filed Oct. 27, 2004, entitled “DEVICES AND METHODS TO TREAT A PATIENT”; International Patent Application No. PCT/US05/09322 filed Mar. 19, 2005, designating the U.S. entitled “DEVICE AND METHODS TO TREAT A PATIENT”; U.S. patent application Ser. No. 11/334,105 entitled “METHODS AND DEVICES TO FACILITATE CONNECTIONS BETWEEN BODY LUMENS”; which is a continuation-in-part of U.S. patent application Ser. No. 11/295,281 entitled “OBESITY TREATMENT SYSTEMS”, filed Dec. 6, 2005; which is a continuation-in-part of International Patent Application PCT/US2005/033683 filed Sep. 19, 2005; which is a continuation-in-part of U.S. Non-Provisional patent application Ser. No. 11/148,519 entitled “METHODS AND DEVICES FOR PERCUTANEOUS, NON-PAPAROSCOPIC TREATMENT OF OBESITY”, filed on Jun. 9, 2005 by Michael Gertner, MD; and is also a continuation-in-part of U.S. Non-Provisional patent application Ser. No. 11/153,791 entitled “METHODS AND DEVICES FOR THE SURGICAL CREATION OF SATIETY AND BIOFEEDBACK PATHWAYS”, filed on Jun. 15, 2005; both of which are continuation-in-parts of U.S. Non-Provisional patent application Ser. No. 11/125,547 by Michael Gertner, M.D., entitled “PERCUTANEOUS GASTROPLASTY”, filed May 10, 2005; which is a continuation-in-part of U.S. Non-Provisional patent application Ser. No. 10/974,248 by Michael Gertner, M.D., filed Oct. 27, 2004, entitled “DEVICES AND METHOD TO TREAT A PATIENT”; which claims priority to U.S. Provisional Patent Application Ser. No. 60/556,004 filed Mar. 23, 2004 by Michael Gertner, M.D., entitled “BARIATRIC DEVICES AND IMPLANTATION METHODS”; to U.S. Provisional Patent Application Ser. No. 60/584,219 filed Jul. 1, 2004 by Michael Gertner, M.D., entitled “DEVICES AND METHODS FOR PERCUTANEOUS GASTROPLASTY”; and to U.S. Provisional Patent Application Ser. No. 60/603,944 filed Aug. 23, 2004 by Michael Gertner, M.D., entitled “DEVICES AND METHOD TO TREAT MORBID OBESITY”; and U.S. patent application Ser. No. 11/396,160, filed Mar. 31, 2006 by Michael Gertner, M.D., entitled “EXTRAGASTRIC MINIMALLY INVASIVE METHODS AND DEVICES TO TREAT OBESITY”. All of the above mentioned patents are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to methods and devices for treating heart failure.

### BACKGROUND OF THE INVENTION

Heart failure is a disease reaching epidemic proportions in the United States and the rest of the world. Over 5 million people in the US and 10 million people across the world suffer from heart failure. These numbers are increasing yearly due to improved technology to treat myocardial infarctions and coronary artery disease.

As the heart fails to function properly, it tends to expand over time to compensate for decreased ability to pump blood, leading to further heart failure and creation of a downward spiral ultimately leading to end stage heart failure and death or need for a heart transplant.

Proposed solutions to prevent cardiac dilation involve placement of meshes or nitinol sleeves (so called restraint devices) over the epicardium to prevent dilation and prevent the downward spiral. These devices are placed around the heart to apply a pressure on the heart wall. Because they are placed around the heart, an increase or decrease in tension on one region of the constraint device translates to an equal increase in tension on another region of the heart. Like a belt, an increase in the tension on one side causes an equal increase in tension on the other side of the belt. This is a major limitation of these devices because the right side cannot tolerate too high a pressure or it will be unable to fill. A further limitation of these devices is that they are not adjustable (reversible or titrateable) and are not removable from around the heart once they are placed because the materials that are used to produce these devices can induce tremendous scarring and inflammation. Furthermore, a major surgery is required to place them around the myocardium (stemotomy or thoracotomy).

Anatomically, the heart has four primary layers, the endocardium (blood contacting surface), the myocardium (muscle), the epicardium (the shell just outside the myocardium), and the pericardium (the outer covering of the heart). There exists a potential space between the pericardium and the epicardium (pericardial space) which can be filled with fluid. Sometimes, the fluid pressure is too high in the acute setting and the heart cannot expand. Typically, when the fluid pressure is uniform, the vena cava, the right atrium, and the right ventricle are the first structures which are compromised. These structures are compromised at pressures of about 10-20 mm Hg and a volume of less than 100 cc in the acute setting.

### SUMMARY OF THE INVENTION

The present invention includes devices and systems which are capable of applying pressure and constraint to the heart and use the pericardium to assist in the application of the pressure and force to the heart.

Aspects of the invention relate to a method of managing a heart failure patient. Anatomically, the patient has skin, costal cartilage, xiphoid and a heart. The heart has a left ventricle, a right ventricle, a left atrium, a right atrium, an epicardium, a pericardium and a pericardial space between the epicardium and the pericardium. The method of managing heart failure in the patient includes placing a support structure between the epicardium and the pericardium such that a force is transmitted from the pericardium through the support structure to a selected region of the epicardium, and leaving the support structure in place between the epicardium and the pericardium postoperatively.

The method step of placing the support structure may include placing a guide wire into the pericardial space through a puncture in the skin, positioning the guide wire over a region of interest of the heart, delivering the support structure over the guide wire to the pericardial space; and removing the guide wire. In some embodiments of this method involving placing a guide wire, the support structure includes a plurality of separate segments, and the plurality of separate segments is delivered over the guide wire one at a time. Some of these embodiments may further include interconnecting the separate segments after they are delivered over the guide wire. And in some of these embodiments, the separate segments are interconnected by joining-magnets located on the segments. In some embodiments, the segments are connected by elastic joints and are stretched longitudinally through a port, thereafter being delivered segment by segment out the distal end of the port.

The method step of placing the support structure may include placing a flexible sheath into the pericardial space through an opening in the skin, positioning the sheath adjacent to a region of the heart to support, delivering the support structure through the sheath to the pericardial space, and removing the sheath. In some embodiments of this method step, the sheath is placed through an incision made in close proximity to the xiphoid. In other embodiments, the sheath is placed through the ribs; in other embodiments, the sheath is placed underneath the sternum from a small neck incision.

In some embodiments of the method of managing a heart failure patient, the support structure may further include an extrapericardial extension and wherein the extrapericardial extension further includes at least one securing portion that secures the support structure outside the pericardium.

In some embodiments of the method of managing a heart failure patient, the method may further include securing the support structure in place without sutures. In other embodiments of the method, the support structure is delivered through an opening in the pericardium no larger than about 1.5 cm. And in still other embodiments, the pericardium is maintained substantially intact while placing the support structure. In other embodiments, clips, sutures, locks, meshes, bolts, and/or fasteners are used to secure the device to the pericardium.

In some embodiments of the method of managing a heart failure patient, the support structure is expandable, and some of these embodiments, the support structure is expandable with a fluid. In some of these fluid-expandable support structures, the fluid expandable support structure is set at the time of implantation to reach a pressure of less than about 20 mm Hg when the heart is expanded during diastole. In some embodiments where the support structure is expandable, support structure is constructed to substantially cover one of the ventricles but not the other. In some embodiments, the fluid is water or saline; in some embodiments, the fluid is a gel; in some embodiments, the fluid is a gas; in some embodiments, the fluid is a curable gel.

In some embodiments of the method of managing a heart failure patient, the support structure applies a force substantially only to the left ventricle and not to the right ventricle.

In some embodiments of the method of managing a heart failure patient, the method further includes the step of adjusting said support structure such that said support structure transmits less than 30 mm Hg to the selected region of the epicardium through transfer of force from the pericardium through the support structure to the selected region of the epicardium. In some of these embodiments, the selected region of the epicardium is the left ventricle. In some embodiments, the selected region of the epicardium includes at least a portion of one of the atria.

Some embodiments of the method of managing a heart failure patient further include removing the support structure. Some embodiments further include adjusting the support structure to modulate a therapeutic effect of the support structure. And in some embodiments, the structure further includes an electrical conducting portion and said electrical conducting portion is activateable after implantation to create a desired therapeutic effect. In other embodiments, the support structure transmits other types of energy such as RF, ultrasound, light, heat, mechanical waves, suction, vibrations, and/or microwaves. In some embodiments, a subcutaneous port attached to the device through a connector in the pericardium stores energy and sends energy to the support structure. The energy can also be used to power an electronic circuit associated with the port for pacing and sensing applications.

The invention further relates to a method of cardiac treatment that includes providing an implantable insert having an inflated state and a deflated state, placing the insert into a patient's body in the deflated state and positioning the insert over a selected region of the patient's heart, inflating the insert to put pressure on the selected region without applying substantial pressure to other regions of the heart; and maintaining pressure in the insert for an extended period of time to create a desired therapeutic effect on the region.

In some embodiments of the method of cardiac treatment, the desired therapeutic effect includes inhibiting cardiac dilation of the region.

In some embodiments of the method of cardiac treatment, the insert is placed between the epicardium and the pericardium.

Some embodiments of the method of cardiac treatment include deflating and removing the insert. In various embodiments of this method, the pressure is maintained in the insert for at least 1 month before removal. In the embodiments, pressure is maintained for at least 6 months before removal. And in still other embodiments, pressure is maintained for at least 2 years before removal. In some embodiments, the pressure within the insert is optimized, titrated, removed, and/or inserted over time through percutaneous access of the port through the skin.

In some embodiments of the method of cardiac treatment, pressure is maintained in the insert such that it does not exceed a maximum of about 5 mm Hg, in others it does not not exceed a maximum of about 25 mm Hg, and in still others, it does not exceed a maximum of about 40 mm Hg. In some embodiments of the method, a minimum pressure of about 5 mm Hg is maintained in the insert, in others a minimum pressure of about 10 mm Hg is maintained in the insert, in others a minimum pressure of about 20 mm Hg is maintained in the insert, and in still others a a minimum pressure of about 5 mm Hg is maintained in the insert. In some embodiments, the pressure inside the insert is sensed using a pressure sensor associated with the support structure.

In some embodiments of the method of cardiac treatment, the selected region is the left ventricle.

The method of cardiac treatment may further include placing a reservoir port in the patient's skin and changing the pressure in the insert by adding fluid to or removing fluid from the insert through the port.

The method of cardiac treatment may further include placing a fluid pump in the patient's body in fluid communication with the insert to automatically pump fluid into the insert upon external command or through an internal feedback system.

The method of cardiac treatment may still further include placing at least one sensor in the patient's body for monitoring a parameter associated with the insert. In some embodiments the parameter is pressure, in others it's strain, in still others it's volume.

The invention further relates to a system for treating a heart; the system includes an inflatable support structure configured for implantation in a pericardial space, the support structure configured to transfer a force from the pericardium through the support structure to a selected region of the epicardium, the support structure further configured to be deliverable to the pericardial space through an opening in the skin no larger than about 1.5 cm; and an implantable tube in fluid communication with the support structure for providing fluid to inflate the support structure.

In some embodiments of the system for treating a heart, the support structure includes a plurality of compartments, each compartment being inflatable to a different pressure. In other embodiments, the system further includes a plurality of inflatable support structures. In some of these embodiments, the system further includes a plurality of implantable tubes, each of the tubes in fluid communication with one of the support members.

In some embodiments of the system for treating a heart, the support structure includes a pacing electrode configured to contact the epicardium.

Some embodiments of the system for treating a heart further include an implantable fluid pump in fluid communication with the implantable tube for delivering fluid to the support structure. Still others include an implantable sensor.

Some embodiments of the system for treating a heart further include an implantable system which inflates or deflates the support structure based on measureable parameters related to the support structure.

In some embodiments of the system for treating a heart, the support structure includes a composite material. In some of these embodiments, the composite material includes an elastomer and a second material to create a curvature to conform to the heart shape. In some embodiments, the composite material includes an elastomer and a coating. In some embodiments, the coating includes a hydrophilic coating, and in some embodiments the coating is a fibrosis-inducing coating. In other embodiments, the coating is conductive and is configured to transmit electrical energy to the epicardial surface. In some embodiments, the coating is different on different regions of the support structure. In other embodiments the composite material includes a shape memory alloy. In other embodiments, the support structure with a composite material includes a coating with a pharmaceutical molecule attached, and in still others, the support structure is configured to release pharmaceuticals.

The invention further relates to a heart-restraining device that includes an expandable support configured to deploy from an access sheath smaller than about 2 cm into a pericardial space around a heart, the support configured to expand into a heart-restraining configuration upon instillation of a fluid into the support, the support further configured to be implanted for an extended period of time to restrain the heart.

In some embodiments of the heart-restraining device, the expandable support is configured to encircle a portion of the epicardial surface of the heart. In other embodiments, the expandable support includes a composite material. In some of these embodiments, at least one portion of the composite material induces a shape change in the support. In other embodiments the composite material induces a desired biologic effect around said device.

In some embodiments of the heart-restraining device, at least one portion of the support is adapted to transmit energy to a portion of the heart or pericardium.

In some embodiments of the heart-restraining device, the support has a width and a thickness, and wherein the width is at least two-fold greater than the thickness, in others, the width is at least five-fold greater than the thickness, and in still others, the width is at least ten-fold greater than the thickness.

The invention still further relates to a heart restraining device that includes an expandable support configured to deploy from an access sheath smaller than about 2 cm into a pericardial space around a heart, the support configured to expand into a heart restraining configuration upon instillation of a fluid into the support, the support further configured to be implanted for an extended period of time to restrain the heart, and at least one radio-opaque marker permitting the device to be visualized from outside of a body.

Some embodiments of the heart restraining device include a plurality of radio-opaque or otherwise visualizeable markers located on, in or around the expandable support.

In some embodiments of the heart restraining device, the marker can be detected fluroscopically.

In some embodiments of the heart-restraining device, the support has a width and a thickness, wherein the width is at least two-fold greater than the thickness. In some of these embodiments, the width is at least five-fold greater than the thickness, and in others, the width is at least ten-fold greater than the thickness. In some embodiments, the support structure is produced from a membrane less than 200 microns thick. In some embodiments, the support structure is produced from a membrane less than 50 microns thick. In some embodiments, the support structure is produced from a material less than 25 microns in thickness. In some embodiments, the support structure is produced from a polyurethane, a silicone, PTFE, or combinations thereof.

In one embodiment, the device is implantable in the pericardial space through a sheath and through a small incision or puncture just under the xyphoid bone. In another embodiment, the device is implantable percutaneously through a small incision or puncture between the ribs.

In one embodiment, the device can be expanded with a fluid; after insertion into the pericardial space, the device in this embodiment is expanded to fill a selected space and volume in the pericardial space and also to apply a pre-determined pressure to the epicardium. As the heart expands and contracts, pressure builds inside the device, transmitting pressure from the pericardium to the myocardium and to the epicardial surface of the heart.

The device can be part of a system in which the device is adjustable through a reservoir port in the skin. Adjustment can be made either percutaneously with a needle to insert fluid into the port and then into the device, or through a transmitter which signals a mechanical pump to initiate pressure/volume adjustment of the device. The reservoir allows for titration of the volume/pressure inside the device and can also act as a module for sensing or other smart electronics related to the implanted devices.

One or more devices can be placed inside the pericardium. The one or more devices can be placed at different regions on the epicardial surface of the heart. The one or more devices can exert different independent forces on the epicardium through modifications of the material properties of the inserts or through differing volumes inside the devices. The one or more devices exert a force on the heart when the heart expands against the device and the device pushes on the inside surface of the pericardium.

The force(s) which are created by the devices are a combination of hydrostatic and material forces. That is, as the implant is compressed by the expanding heart (during diastole), the pressure inside the insert increases because of the tensile properties of the device. The hydrostatic pressure within the insert exerts a normal force on the epicardium. In addition, as the heart contracts during systole, the already expanded insert contracts down and exerts the stored potential energy on the myocardium.

In one embodiment, the device(s) augments the natural pericardial constraint applied by the pericardium, therefore acting as a composite material in combination with the pericardium to restrict expansion of the heart. In some embodiments, the device(s) are elastic, expanding during the diastolic cycle of the heart and contracting with the systolic cycle of the heart to exert a restrictive force during diastole and a corresponding compressive force during systole as the elastic potential energy leaves the device material.

The material used to manufacture the device is important. In some embodiments, the insert is produced from a hydrophilic material which absorbs greater than 10 percent water. In some embodiments, the hydrophilic material absorbs greater than 50 percent water and in some embodiments, the hydrophilic material absorbs greater than 90% water. In some embodiments, the insert material can absorb up to 99% water. By absorbing water, the material interface with the epicardium is lubricious and advantageous in some embodiments. In some embodiments, the material is biodegradeable. For example, in some embodiments, the material is biodegradeable over about a 4 week period. In some embodiments, the material is biodegradeable over about a three month period. In some embodiments, the material is biodegradeable over about a six month period. In some embodiment, the material is biodegradeable over about a one year period. In some embodiments, the material is biodegradeable in a two year period or less. In some embodiments, the material is biodegradeable upon photo-activation or another energy source.

In some embodiments, the device can be attached to one or more fill lines (e.g. tubes) operable to fill the one or more pericardial devices with a fluid. The fill line(s) can be permanently or temporarily implantable. The fill line(s) can be connected to one or more reservoirs to create a closed system with fluid inside both the insert and the reservoir. The reservoir(s) can be chronically implanted and/or fillable via puncture through the skin. Alternatively, the reservoirs can be automatically inflated or deflated with small pumps implanted under the skin in communication with the reservoirs. The pumps can be manually operated or automatically operated from within the patient or external to the patient.

In some embodiments, the devices can exert different surface forces on different regions of the heart by virtue of being composed of different materials or different material configurations; in some embodiments, the individual devices contain different amounts of fluid. In some embodiments, it is an object to control the pressure inside of the inserts. For example, it may be desirable to maintain the maximum pressure within the pericardial inserts to less than 25 mm Hg or in some cases to less than 15 mm Hg.

In some embodiments, a relief valve is provided on the fill lines so that a maximal pressure, if exceeded, triggers valve opening and release of hydrostatic pressure within the device.

The devices can be made from individual parts in some embodiments. For example, the inserts can be connected to one another by rigid or semi-rigid connectors which may or may not be fillable with fluid. The inserts can be connected by magnetic connectors in some cases. The magnetic connectors allow for self-assembly of the inserts inside the pericardial space.

In some embodiments, the device(s) can have integral sensors through which physiologic parameters are measured. For example, the sensors can measure the hydrostatic pressure inside the inserts or the hydrostatic pressure inside the pericardium. The sensors can measure the stress or strain on the inserts, on the surface of the heart, or on the inner surface of the pericardium. The sensors can detect electrical activity on one or more regions of the heart. The sensors can be placed on or in the devices, on the fill lines, on the reservoirs, or on any other structure attached to the inserts. Any or all of the sensors can communicate their data to a remote receiver or to one another.

When the physiologic sensor detects that the pressure is outside the desired range, the patient or physician can be alerted. In some embodiments, an automated pump is activated and fluid is added or removed from the device(s).

The inserts can be associated with electrodes. The electrodes can be integrally attached to the inserts or they can be attached directly to the epicardium or pericardium. The electrodes can communicate with the epicardium or other parts of the autonomic nervous system such as the parasympathetic or the sympathetic nervous systems. The electrodes can communicate with the sensors to create a communication or feedback circuit. Additional electronics, sensors, actuators, electrodes, and computer software can also be incorporated into the system. Radiofrequency transmitters can also be employed to relay information to the patient or to physicians involved with the care of the patient.

In some embodiments, the inserts are attached to the inner portion of the pericardium and in other embodiments, the inserts are attached to the epicardium or myocardium. Attachment can be achieved with sutures, with glues, or via tissue ingrowth into the electrodes. In some embodiments, energy, such as radiofrequency, microwave, or laser energy can be used to attach the devices to the pericardium or epicardium.

In some embodiments, the devices are elastic and expand as pressure is created within them.

In other embodiments, the device(s) are folded up for insertion into the pericardial sac through a sheath. In some embodiments, the devices are created such that they can be stacked longitudinally within a sheath and placed inside the pericardium one segment at a time.

In some embodiments, the device(s) are folded and placed in a sheath and then expanded when they are placed inside the pericardial space.

In some embodiments, the device(s) are linked to one another or conform to the shape of the heart such that when expanded the inserts create a restrictive force on the myocardium.

In some embodiments, the device(s) are used to treat one or more atria.

In some embodiments, the device(s) are used to treat one or more ventricles.

In some embodiments, the device(s) are used to treat atria and ventricles.

In some embodiments, one or more parameters of the inserts is measured over time (e.g. pressure or tension) and the volume of the insert is adjusted based on this recording. In some embodiments, the volume is adjusted by a physician and in some embodiments, the volume is adjusted automatically by an implanted pump. One example of an implanted pump is a piezo-electrically actuated pump.

In some embodiments, a fluid such as saline is used to fill the expandable device(s). In some embodiments, a gas such as carbon dioxide, nitrogen, xenon, or air is chosen. In some embodiments, a fluid such as a hydrogel is used. In some embodiments, a pharmaceutical compound is included in the fluid in the insert and is slowly released into the pericardial space.

In some embodiments, a method is described in which the pressure or volume of fluid inside the inserts is adjusted based on measured tension on the skin of the inserts.

In another embodiment, a hydrophilic material is used for the skin of the insert and the hydrophilic material can absorb some of the fluid within insert. Examples of a hydrophilic material is polyurethane. Another hydrophilic material is cellulose or bacterial cellulose. In another embodiment, a primary material such as a polyurethane or PTFE is used and a hydrogel coating such as a poly-ethylene glycol (PEG) placed on the primary material as a coating.

In some embodiments, a hydrophobic material is used for the skin of the insert.

In another embodiment, the insert has a porous or semi-porous material in which gas or materials can permeate. In some embodiments, the material is hydrophilic and gas permeable.

In another embodiment, a system is described in which the inserts comprise sensors and the information from the sensors is transmitted through the skin of a patient to a receiver outside the patient.

In some embodiments, the sensors cover the inserts or are placed inside the implants. In some embodiments, the sensors reside in the attached tubing or port or are hydraulically associated with the attached tubing or ports.

In some embodiments, the inserts are placed through a pericardial window or through a thoractomy or through a catheter placed into the heart via blood vessels.

In some embodiments, the inserts are coated with a material such as parylene, silicone, Dacron to alter the interaction between the surface of the insert and the epicardial surface.

In one embodiment, a method is described in which pressure in the insert is measured periodically from 1 day to 60 days depending on the patient and physician. In some embodiments, it may be desirable to adjust the pressure within the insert every 60-120 days. The desired pressure inside the pressure sensor may be less than 10 mm Hg or less than 20 mm Hg or less than 30 mm Hg. In some embodiments, a pressure between 10 and 20 mm Hg is desired and in some embodiments, a pressure between 0 and 10 mm Hg is desired. In this method of use, the pressure in the insert is obtained via internal or external pressure; the pressure in the insert is then adjusted by adding or removing fluid from the insert so as to correct the pressure toward the desired pressure.

In another embodiment, an internal pressure sensor complete with data logger is used to continuously monitor pressure within the insert and alert the patient or physician of pressures that are either too high or too low. In some embodiments, the physician or patient then adjusts the pressure in the insert based on the degree of deviation of the pressure from the desired pressure. In some embodiments, the internal pressure monitor communicates with an electrically active device either directly or indirectly. Examples of electrically active devices include drug delivery pumps, pacemakers, defibrillators, resynchronization devices, hydraulic pumps to pump fluid into or out of the inserts.

In another embodiment, a method is described in which a parameter associated with the pericardial insert is measured and the composite material properties of said pericardial insert parameter is adjusted based on said parameter associated with said pericardial insert. In some embodiments, the parameter is hydrostatic pressure. In some embodiments, the hydrostatic pressure is communicated wirelessly through a fluid port.

In another embodiment, a method of delivering a pericardial insert into a pericardial space is described in which said insert is delivered into the pericardium within a sheath, through the pericardial sac, and into the pericardial space. Said sheath is subsequently removed leaving the pericardial insert inside the pericardial space. In some embodiments, said sheath is <5 mm, in some embodiments, said sheath is <1.0 cm, and in some embodiments, said sheath is smaller than 2 mm. In some embodiments, said method further comprises closing a puncture in the pericardium created by the sheath. In some embodiments, said insert further comprises tubing which communicates with said insert and which is operable to fill said insert when fluid is introduced into said tubing. In some embodiments, said method further comprises a sealing ring which fits over said tubing and is operable to hold said tubing in a fixed position within the pericardium without a leak through the pericardium.

In some embodiments, said balloon comprises a first compliant material and a second more rigid and shaped material which defines a specific insert shape.

In some embodiments, the insert comprises integral magnets operable to bring two ends of the insert together; for example, two components of the insert can be placed into the pericardial space and then they can self-assemble via the magnets bringing two pieces of the insert together around the myocardium.

In some embodiments, the insert is filled with a medicament and the medicament (e.g. a nitrate or a beta blocker) is eluted into the pericardial space over time.

In some embodiments, the insert is coated with a material which improves the biocompatibility of the insert by prohibiting ingrowth or preventing effusion formation around the insert. In some embodiments, the insert is coated with a material which promotes ingrowth of fibrous tissue from the pericardium or from the epicardium.

In another embodiment, the insert is removable after a period of time >24 hours. In one embodiment, a material is chosen which resists ingrowth and capsule formation. In one embodiment, this material is a thin polyurethane. In one embodiment, the thickness of the thin polyurethane is less than 50 microns and is preferable less than 25 microns. In one embodiment, the insert material is silicone. In one embodiment, the insert material is a combination of silicone and polyurethane. In one embodiment, the insert can expand up to 100% in volume. In one embodiment, the insert can expand up to 200% in volume.

In one method of use, the insert is implanted for a period of up to 6 months while the myocardium heals after a myocardial infarction; in another method of use, the insert is placed in side the pericardial space for a period of up to 2 years to treat a chronic dilated heart.

In one embodiment, the insert encircles the myocardium and provides for circumferential support. In another embodiment, the insert rests against a portion of the myocardium to support a region of the heart.

In one embodiment, the pressure within the insert is maintained at a maximum of about 10 mm Hg. When lOmm Hg is exceeded, a receiver external to the patient is alerted. In some embodiments, this maximum pressure is about 20 mm Hg. In some embodiments, the maximum pressure is about 30 mm Hg. In some embodiments, the maximum pressure is about 40 mm Hg. In some embodiments, the maximum pressure is about 5 mm Hg.

In one embodiment, a system for controlling expansion of the heart comprises a nozzle operable to be inserted into the pericardium for introducing fluid into the pericardium in a controllable manner; a fluid line connected to the nozzle; a port coupled to the fluid line and adapted to be accessed so as to inject fluid through the fluid line and through the nozzle into the pericardium. In another embodiment, the system comprises a reservoir to store fluid. In another embodiment, the system comprises a sensor to sense pressure in the pericardium or sense another parameter related to the heart. In another embodiment, the port comprises a pressure sensor. In another embodiment, the system comprises an actuator to push fluid into the pericardium through the nozzle. In another embodiment, the system flurther comprises a fluid expandable structure. In another embodiment, the fluid expandable structure comprises a balloon.

In one embodiment, a method to treat heart failure is described in which fluid is placed into the pericardium or taken out of the pericardium through a port and fluid line. The pressure is sensed and the patient is assessed and fluid is again removed or placed into the pericardium to create a pressure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 depicts an insert with more than one compartment or a common compartment connected via a valve. Reservoir communicates with the inserts via tubing and the inserts are fillable through the tubing and/or through the reservoir

FIG. 2 depicts an example in which there is no insert and the fluid reservoir communicates with the pericardial space and the pericardial space is filled with free fluid rather than an insert. The space remains fillable through the reservoir and the pressure can be maintained/controlled using the subcutaneously implanted reservoir.

FIG. 3 depicts a system in which the insert comprises electrodes or magnets or sensors within the inserts. The inserts are fillable through fluid lines and the sensors/magnets/electrodes are controllable or detected through the port reservoir connected to the inserts.

FIG. 4 depicts the insert outside of the pericardium. A coating is depicted on the insert. The coating can control the biologic reaction to the insert.

FIG. 5 depicts an insert which can self-assemble into a structure inside the pericardium.

FIG. 6 depicts a method for deploying an insert into position.

FIG. 7 depicts an insert being deployed.

FIG. 8A depicts an insert positioned between the epicardium and the pericardium in a deflated state.

FIG. 8B depicts an insert positioned between the epicardium and the pericardium in an inflated state.

FIG. 9 depicts a subcutaneous access port and an insert positioned to exert pressure only on the left ventricle.

FIGS. 10A-10B are various views depicting one insert embodiment.

FIGS. 10C-10E are various views depicting another insert embodiment.

FIGS. 11A-11C are various views depicting another insert embodiment.

FIGS. 11D-11F are various views depicting another insert embodiment.

FIG. 12 depicts an example in which there is no insert and the fluid reservoir/port communicates directly with the pericardial space.

### DETAILED DESCRIPTION OF THE INVENTION

Pericardial Insert

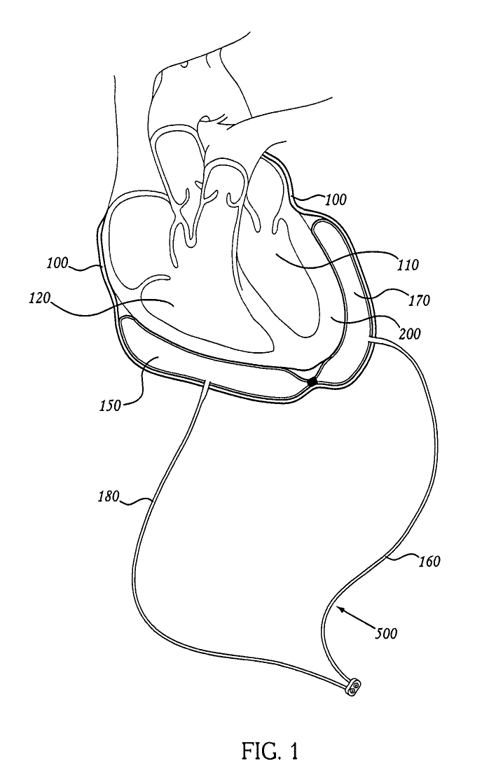

FIG. 1 depicts a heart 110, a pericardium 100, and an insert 150,170 inside the pericardial space. The inserts 150, 170 or single insert (e.g. 150) are placed inside the pericardium to support a wall of the heart (e.g. the left ventricle) to prevent the wall from dilation, remodeling, or otherwise exhibiting maladaptive behavior; the insert may be placed as a chronically implanted insert (e.g. for 1-2 years), a sub-acute insert (e.g. 3 months in a dilated heart), or as an acute insert (e.g. immediately after a myocardial infarction and for 3-6 months thereafter).

In some embodiments, the insert can hold a fluid or gas within its walls; in some embodiments, the insert can be a solid material. One or more inserts inside the pericardium can be filled with a fluid (liquid or gas) and the level of fluid or pressure inside the insert can be adjusted; if more than one insert is in place, then the fluid and/or pressure in each can be independently adjusted in the one or more inserts. The one or more inserts 150, 170 can exert a force on the heart when the heart expands against the insert.

The pericardium 100 creates pressure on the insert when the heart expands; the pressure inside the insert in turn results in pressure on the myocardium 200. Pressure on the myocardium, as discussed above, can result in reversal or prevention of the maladaptive remodeling process. In the embodiment where inserts 150, 170 inside the pericardium are fillable with a fluid, the remodeling force can therefore be adjusted over time through fluid lines 160, 180 which are accessible through a port such as subcutaneously implanted port 130. The material can be a thickened polyurethane or thickened silicone elastomer which supports the heart via force transduction through the wall.

In some embodiments, inserts 150, 170 provide different forces to different regions of the epicardium. In one example, insert 170 is separated from insert 150 by a flow restrictor 190. Flow restrictor 190 can restrict the rate at which fluid may pass between inserts 150 and 170. In some embodiments, flow restrictor 190 may completely block fluid flow between inserts 150 and 170. In the above embodiments, compartments 150 and 170 are independent. For example, insert 170 can apply a greater or lesser force to the epicardium/myocardium than insert region 150. The forces applied to the heart can be different due to different hydrostatic pressures inside the one of the insert regions or as a result of different materials used to produce the inserts. For example, the insert can be made from a thicker material on one side of the heart and a thinner material on the other side of the heart. When the heart expands during diastole, the side with the thicker material will result in a higher force applied to that region of the heart.

Pressures inside different regions of the insert or inserts can also be controlled independently through fill tubes 160, 180 which communicate with the insert regions 150, 170. Different forces can be applied to the different regions of the epicardium/myocardium depending on the amount of volume placed inside the insert(s) or inside different regions of the insert. For a given volume inside the insert, the myocardium will experience a given pressure. With a greater volume, the myocardium will experience greater pressure; for less volume, the myocardium will experience less continuous pressure. Such adjustability or titrateability is advantageous over time because the remodeling forces may need to be modified over time as the heart dilates or contracts.

In some embodiments of the invention, insert volumes may range from 10 cc to 90 cc or from 2 cc to 100 cc. In some embodiments, the desired volume is between 100 cc and 200 cc of fluid. A range of viscosities may be chosen for the fluid inside the insert. For example, water or saline solution may be the desired fluid inside the insert. Such fluids may have viscosities of around 0.75 to 1.25 cP (centipoise). In some embodiments, fluids such as dextran or other solutions containing a liquid and a larger molecule may be used. In some embodiments, the fluid is not homogenous and has a liquid phase and a solid phase, or a liquid phase and a gas phase. In some embodiments, it may be desirable to have a fluid with viscosity greater than 1.25 cP (for example, in the range from 1.25 to 100 cP). In some cases, thick fluids may be desired which have viscosities greater than 100 cP (for example, 101 to 1500 cP). For example, glycerol has a viscosity of 1490 cP. In some scenarios, it may be desirable to have a fluid with a viscosity less than 0.75 cP, for example down to 0.05 or 0.10 cP. In some scenarios, it may be desirable to use a gaseous fluid such as air, nitrogen, carbon dioxide, xenon, oxygen.

The inserts 150, 170 can be shaped or a material can be chosen so as to exert a force on a pre-determined area of the epicardium; alternatively, the material can be chosen so that as the heart expands during diastole, the inserts are compressed and the compression pressure expands the material of the insert (dependent on the material properties of the insert); the increased pressure inside the insert is also transmitted to the myocardium. The pericardium therefore acts to constrain the heart and the constraint is modified by the material properties of the insert. The material properties of the insert in turn may be modified by the filling status of the insert in the embodiment when the insert is fillable.

Methods of Manufacture:

As an example, an insert was manufactured using a polyurethane blend (e.g. hydrothane 93A) from CardioTech International. An insert was created using this material by placing formed pellets (the way the material is sold by the manufacturer) into THF or DMAC (a solvent). A mold in the desired shape of the insert is then used to shape the implant; the mold is dipped into the hydrothane-solvent solution and dried to create the elastic insert which will be placed into the pericardial space. The neck of the insert may be defined by the mold or the the insert may be manufactured with a wide mouth and then crimped over the fluid communication lines which communicate between the port and the insert.

In another manufacturing embodiment, the sheet of material is wrapped around a mold and the ends are heat sealed so as to create an enclosed volume. The balloon in this or any of the embodiments can be further modified such that the different regions are created by sealing different regions of the balloon so that it looks like an air-mattress when it is inflated.

The insert(s) 150, 170 may be ribbed or have many small bubbles along its surface. Such raised areas can ensure a relatively uniform distribution of pressure along the myocardium. One or more ribs can have greater or less thickness than the other ribs so that the compliance can be varied over the surface of the insert. In addition, the pressure within each rib or bubble can be adjusted independently over time.

One or more independent inserts 150, 170 may be placed within the pericardium, each with its own compliance and material properties. For example, inserts 150, 170 may possess different material properties, sizes, or thicknesses so that the insert exerts less force on the right ventricle than the left ventricle or vice-versa (as an example). In some instances, inserts 150, 170 are placed close to a region of the left or right atria so as to decrease the amount of stress on the atria to treat and/or prevent arrhythmias by allowing the atria to decrease in size. In some embodiments, the inserts are connected to one another within the pericardium by a connector 155 which links the inserts to one another. In some embodiments, the connector 155 acts as a fluid conduit between the pericardial inserts 150, 170. In some embodiments, a magnet is incorporated into the insert and acts as the connector by bringing components of the insert together inside the pericardium. Magnets placed inside of the inserts can also facilitate attachment of one or more inserts to one another. Magnets may be incorporated into the material of the inserts or may be secondarily attached with a glue to the inserts.

In some embodiments, one or more valves 155 are placed between the two inserts and the inserts are fluidically connected by the valve. The valve or valves can be opened or closed depending on the relative pressures within each of the inserts 150, 170. The valve or valves may be passively controlled based on pressure or may be actively controlled depending on the relative pressures inside the inserts. The valves may be controlled from a region external to the patient through a wireless transmitter. In some embodiments, the valve is a flow restrictor between the inserts, preventing or limiting the amount of flow between the inserts.

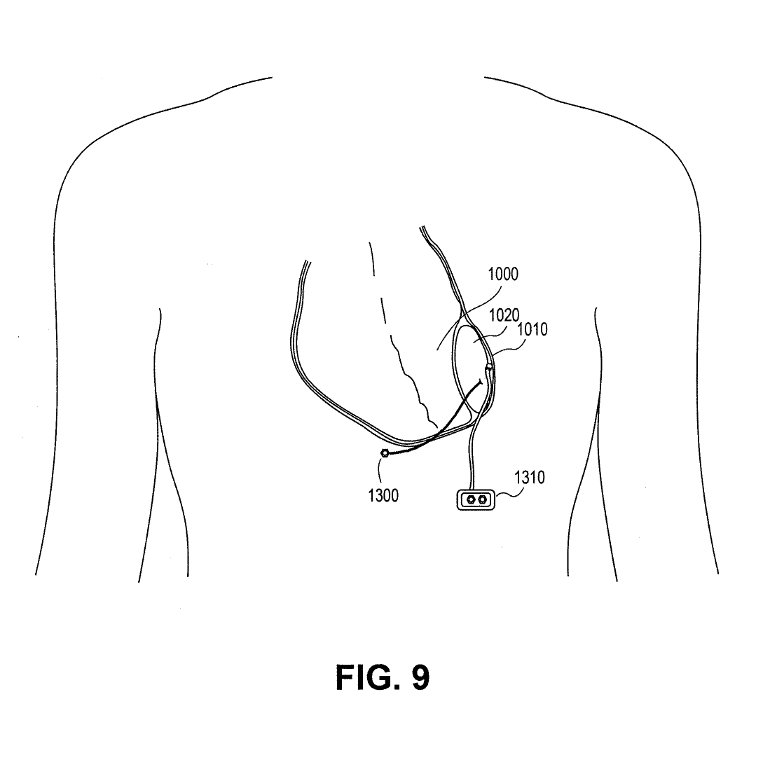

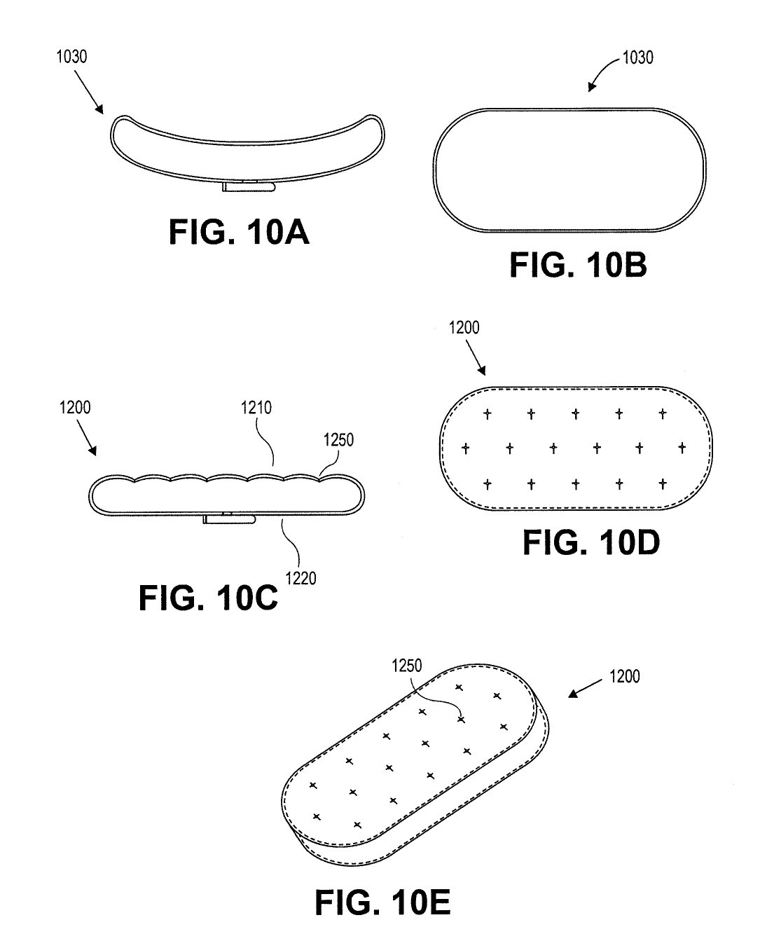

In some embodiments of this invention, only one insert is placed inside the pericardium. For example, an insert 1020 is placed between the left ventricle 1000 and the pericardium 1010, such as shown in FIG. 9, and exerts pressure only on the left ventricle 1000. This allows the right ventricle to continue to expand freely against the pericardium 1010. The single insert 1020 can be shaped in a way to optimize force on the myocardium. For example, one shape is a C shape or a crescent shape which can grip the heart and apply a directional force, such as insert 1030 shown in FIGS. 10A and 10B. Another shape (not shown) is shaped like a baseball glove to hold the heart inside. Another shape (not shown) is a malleable shape in which the pericardium and myocardial forces shape the insert rather than the insert having a baseline shape. Another form is that of an air mattress 1200, such as shown in FIGS. 10C-10E. In this shape, the side 1210 facing the heart has a bubble contour 1250 which can more naturally fit the contour of the heart.

Pressure Control of the Inserts

Pressure within the inserts can also be controlled by a valve external to the insert. In FIG. 1, the valve or port 130 is implantable subcutaneously. In one embodiment, the valve is a reservoir with a membrane. The membrane can be a silicone membrane which is accessible through the skin with a needle. The needle punctures through the skin and then through the membrane; the silicone can self-seal after the needle is removed. The reservoir and membrane create a valve, the valve being accessed and “opened” when the needle passes through the membrane.

In this embodiment, the pressure or the force on the ventricle is maintained by the constant volume inside the insert-port system. The pressure or force can therefore be adjusted through adjustments in the volume in the system and the adjustments are performed by accessing the port and injecting fluid into the system or removing fluid from the system.

In further embodiments, the volume in the system is adjusted automatically through an implanted pump (as one example). The implanted pump communicates with the system and adjusts the volume in the system automatically.

In some embodiments, at least one sensor is provided which communicates with the insert or inserts. In this embodiment, the sensor is a pressure sensor, a strain sensor, a motion sensor, an accelerometer, a position sensor, a capacitance sensor, a resistive sensor, a temperature sensor, a pH sensor, or any other type of sensor which detects a physiologic change on the insert.

Other examples of sensors are electrical sensors which sense currents or other electrophysiologic parameters. Sensors can be placed on the epicardium or within the myocardium to detect any of the physical or electrophysiologic parameters described above.

The sensors can send signals through the patient to an external receiver or the sensor can send the signal to an internal storage unit for download to an external unit at a later time. The internal storage unit can store and interpret the signals from the sensor. The internal storage unit can communicate with a receiver outside the patient or the internal storage unit can send data to an implanted software program which then can communicate with the automated fluid controlled system. Alternatively, the sensor can communicate with one or more pacing electrode systems on, in, or otherwise in communication with the heart.

Inserts 150, 170 can be connected to supply lines 160, 180, which allow for different amounts of fluid to be placed independently into one insert or the other. These lines can further be attached to a port 130 which enables injection of fluids into the inserts from outside the patient. Port 130 enables physicians to adjust the pressure independently within each insert.

Self-Assembling Inserts

The inserts 150, 170 can include magnets (e.g. sumerium-cobalt or neodymium based alloy magnets). The magnets can be used to increase the force that the inserts apply to the epicardium and/or myocardium. The magnets inside the inserts can also be used to connect one or more inserts during implantation. For example, one or more inserts with magnets can be placed inside the pericardium and the inserts can then self-align within the pericardium when they are placed inside the pericardium so that they create a structure within the pericardium which applies a constraining force to the epicardium and/or the myocardium. The magnets may be placed anywhere inside or outside the inserts. Magnets may be placed anywhere inside the skin of the material. In one embodiment, small magnetic particles are placed inside the material insert or within the fluid inside the insert. In another embodiment, the magnets are placed along the edge of the insert so that the inserts can be held together like pieces of a puzzle.

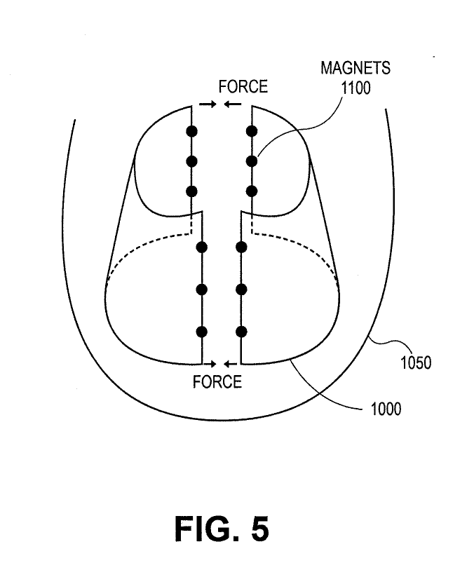

FIG. 5 depicts a self-assembling insert in which magnets 1100 are placed on the edge of the insert 1000. The complex forms a structure inside of the pericardium 1050 through attraction (F) of the magnets and structural components inside the pericardial space.

In one method of implantation, a first contracted, or undeployed portion of the final insert is placed inside the pericardial space, and then a second contracted, or undeployed portion of the final insert, is placed inside the pericardial space, thereafter allowing the individual portions to align or polymerize with one another to form a structure inside the pericardial space. The structure can encircle the heart or can create a force against one portion of the heart. In some embodiments, more than two components of the implant come together to form the insert inside the pericardial space.

Method of Adjustment Over Time:

In one method, the force exerted by the inserts on the epicardium and myocardium is adjusted over time. The adjustment is performed in response to changes induced on the heart by the device. For example, as the heart remodels and its diameter decreases, the force on the myocardium will decrease as there may be more space in between the epicardium and the pericardium; increase in space translates to an increase in volume and a decrease in pressure on the epicardium. Similarly, as the pericardium remodels due to forces exerted on it by the insert, the volume between the epicardium and the pericardium decreases over time. Therefore, in one embodiment of this invention, volume and/or pressure within the insert is adjusted by injecting fluid into the insert. In one example, a subcutaneous port is used to perform these adjustments.

To facilitate adjustment, knowledge of the physiologic force or pressure or other parameter related to the inserts would assist the physician in making decisions. In one embodiment, this information is relayed to the patient or the physician so that decisions can be made based on the information.

In one embodiment, pressure inside the insert is measured over time to quantify the force being applied to the myocardium. In one embodiment, a pressure sensor is placed inside the insert. In another embodiment, a strain gauge is placed inside or outside the insert. These sensors can communicate with the subcutaneous port and then to the patient or physician. Alternatively, the sensors communicate directly with the patient or physician without going through the port. For example, the sensors are placed inside the insert or inside the skin of the insert. In one embodiment, the insert has strain gauges placed on or within the material of the insert.

Insert Shape

Inserts 150,170 can be produced in various shapes including crescent shaped, banana shaped, curvilinear, ring shaped. The inserts may be flat or may be curved with the surface of the heart or pericardium.

Insert Material

The inserts can be made from materials such as PET, PTFE, polyurethane, silicones, or combinations of these materials. In one preferred embodiment, the insert or inserts is made from a highly hydrophilic material such as polyurethane. The inserts can further be coated with hydrophilic coatings so that the insert slides within the pericardium. Another example of a material is a combination material of silicone and polyurethane. Such a composite material allows for the improved elasticity of silicone with the biocompatibility and strength of the polyurethane material.

In some embodiments, the elasticity, or the elongation of the material of the insert can exhibit a strain of 200-300 percent or even up to 500 to 1000 percent. The elasticity determines the spring force with which the insert recoils as the heart begins its contraction phase.

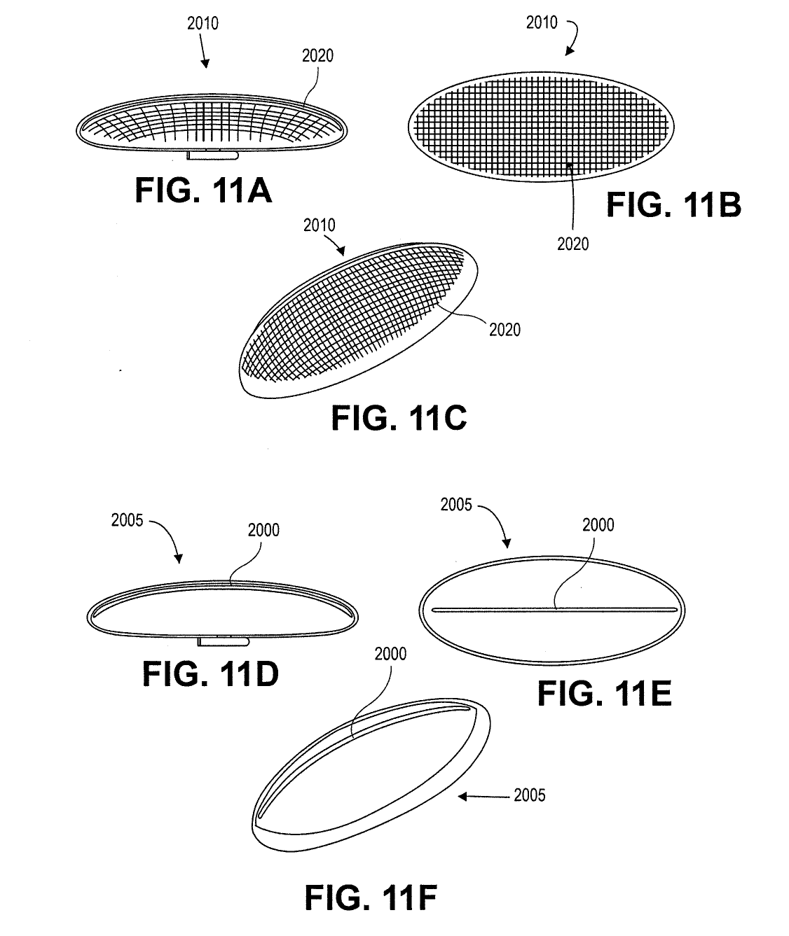

In some embodiments, regions of the inserts can be made more rigid than other regions of the inserts. For example, lines or bars 2000 of a heavier material can be placed on the inserts 2005, as shown in FIGS. 11D-11F so that when the inserts are expanded in the pericardial space, they expand in one direction and remain in place in this direction (e.g. in the longitudinal direction) along the heart wall from cranial to caudal.

In one embodiment of an insert 2010, shown in FIGS. 11A-11C, a composite material is used in which a more rigid material such as polypropylene mesh 2020 or a polyester mesh is used and a second, more biocompatible, flexible material such as polyurethane is molded over the polypropylene.

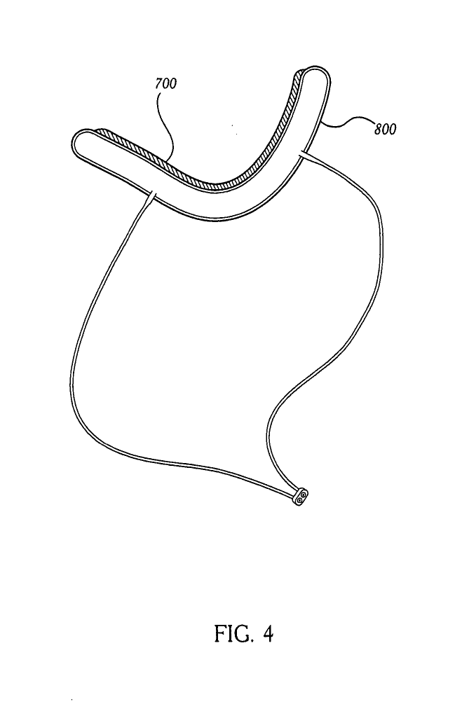

FIG. 4 depicts an insert embodiment in which a composite skin is depicted. A first material 800 and a second material 700 is coated on first material 800. Second material can be a hydrophobic material such as PTFE or a lubricious material such as PVVF. In some embodiments, it may be desired to create a scarring effect between the implant and the outer surface of the heart. In this embodiment, material 700 is a mesh such as polypropylene which can induce ingrowth between material 800 and the epicardial surface.

In another embodiment, the composite material has an insertable or removable component. For example, after the insert is placed in the pericardial space, a second component is placed inside the insert to increase the rigidity or create directionality of the insert inside the pericardium.

In addition to the polymers mentioned above, metals or metal alloys can be used in combination with polymers to support the heart wall. In some embodiments, the materials used need to have a space in which fluid can be placed to create a hydrostatic pressure within the insert. In one example, a fluid fillable insert is made from a polymer such as polyurethane, which in addition has a nitinol mesh as part of the skin of the insert. In another embodiment, the insert has a stainless steel frame as part of the insert to aid in expansion and rigidity of the insert. Other useable metals include cobalt-chrome and titanium.

In any of the embodiments, at least a portion of the insert can be biodegradeable. For example, a coating or the skin of the insert or a part of the skin of the insert can be biodegradeable. The biodegradeable portion can be manufactured so as to degrade over a period of months or years.

In some embodiments, the insert comprises markers or regions for visualization from outside the patient. Such markers are visualizeable via one or more means such as fluoroscopy, MRI, CT scan, and ultrasound/echocardiography.

In some embodiments, the metal is an electrical conductor and the metal can then be used to run electrical current through the metal to interact with the electrical conduction pathways of the myocardium. In one example, a current can be pushed through the material to defibrillate the heart to treat an arrhythmia. In another embodiment, electrical current is run through the material to pace the heart. In another embodiment, electrical current is run through the insert to coordinate contractions of the left ventricle with the right ventricle or with one or more atria to synchronize or coordinate contractions of the heart. In another embodiment, electrical currents are gated to sensors which sense EKG signals. In this embodiment, subthreshold currents are run through the myocardium such as is discussed in (J. Cardiovascular Electrophysiology Vol. 15, pp. 418-427, April 2004 which is herein incorporated by reference).

In some embodiments, the insert is produced in part or in whole from a polymer or non-metallic material which conducts electricity. Current can then be run through the polymer to interact with the conduction pathways of the myocardium.

In another embodiment, electrodes are attached to a region of the heart; these electrodes are run along the pericardial insert while the pericardial insert remains free to float inside the pericardial sac.

Controlled Pericardial Efussion

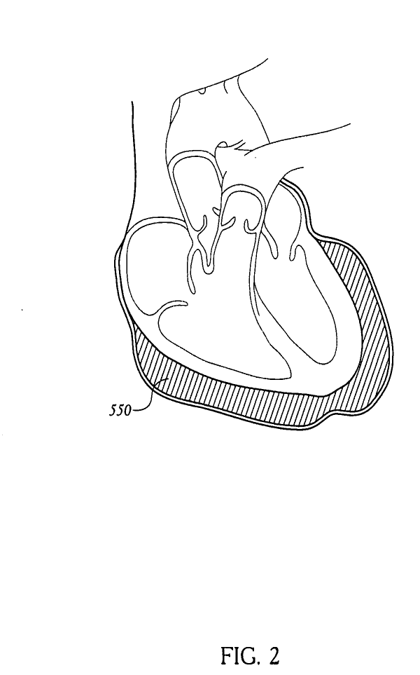

FIG. 2 depicts another embodiment of the current invention in which a fluid 550 is placed into the pericardial potential space without a balloon. The fluid can now freely move inside the pericardial space to exert a hydrostatic pressure on the myocardium. A port such as port 130 described above, may be used in this embodiment to communicate with the potential space so that fluid can be injected and/or removed. Fluids such as saline may be utilized or thicker fluids such as silicone or mineral oil or hydrogels.

The port acts as a valve to control the volume and/or pressure inside the pericardial space. Similar types of sensors can be used as described above. For example, a pressure sensor inside the port can sense the hydrostatic pressure inside the pericardial space and based on this hydrostatic pressure, the amount fluid inside the space can be adjusted.

Combinations with Electrical Modulation

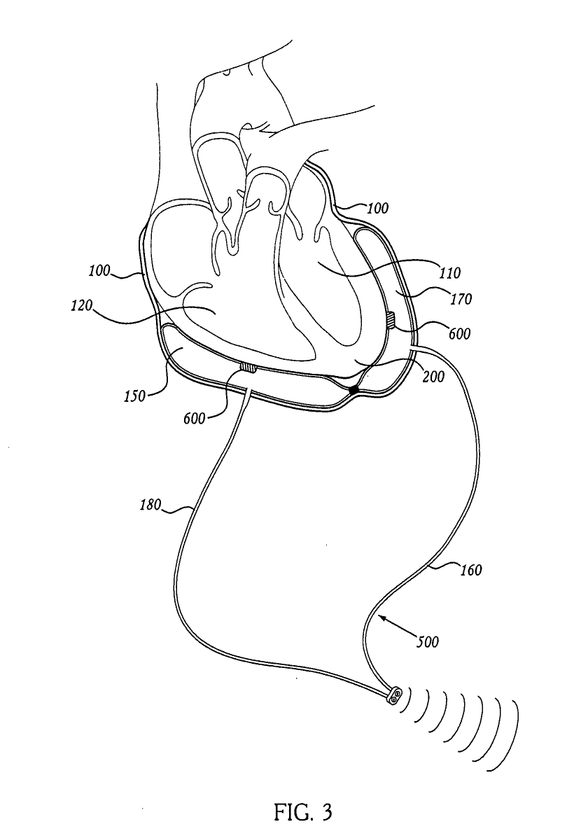

FIG. 3 depicts another embodiment of the present invention in which electrodes 600 are placed on or near the inserts 150, 170. The electrodes work with the insert system to combine beneficial effects of constraint with those of resynchronization, pacing, defibrillation, or any other type of electrical modulation of cardiac tissue. The electrodes are also able to pace the heart or defibrillate the heart. The electrodes can also apply frequencies, currents, waves, and characteristic pulses which do not capture the electrical system of the heart but rather induce remodeling with a sub-threshold set of currents.

Methods of Implantation

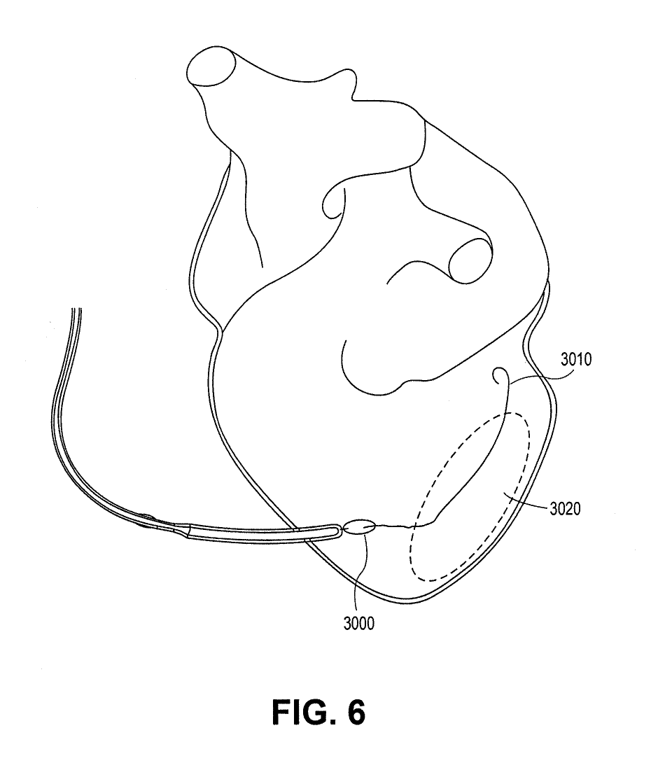

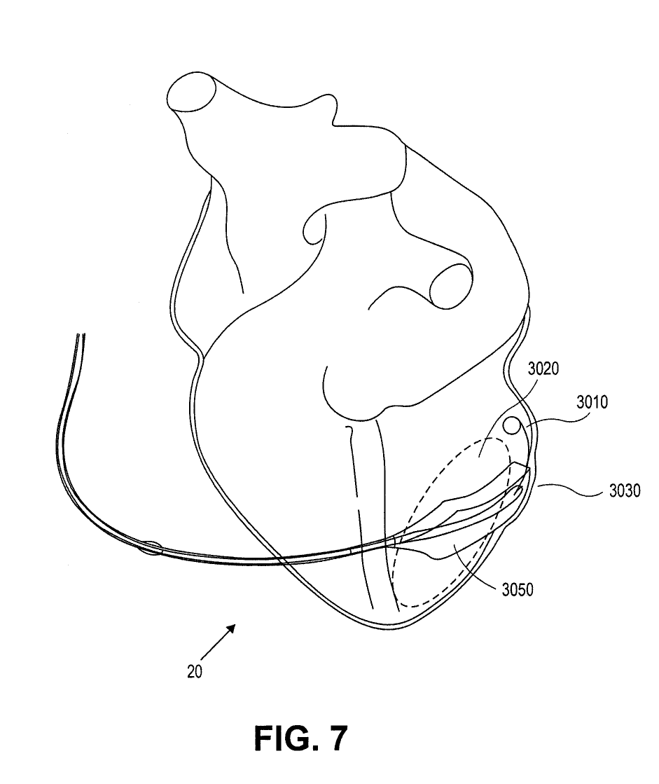

In one method to implant the pericardial inserts, an incision is placed in the skin of a patient and the sub-xyphoid region underneath the inferior sternum is accessed. From this position, the mediastinum can be entered to expose the pericardium. At this point in the procedure, a port can be placed through the skin incision and the port advanced to the pericardium. A camera may be used at this point in the procedure or a fluoroscopy machine can be used to visualize the direction of the port relative to the target region on the epicardium. A small hole (FIG. 6; 3000) may then be made in the pericardium and a camera placed within the pericardial space to visualize placement of the insert. In the case where fluoroscopy is used, a mobile fluoroscopy machine may be utilized to determine the direction of the port and a fluoroscopically visible marker may be placed at the end of the port. The camera may be a CCD camera, a CMOS camera or a fiber optic endoscope. The camera may be placed at the end of a flexible tube or at the end of a rigid tube.

After the camera is placed inside the pericardium and/or fluoroscopy is begun, a guidewire 3010 may be placed inside the pericardium and positioned over the region of the myocardium to be treated.

The insert 3050 may then be advanced over the guidewire 3010 and placed inside the pericardium 3030 between the epicardium 3020 and the pericardium 3030. As described above, the insert can be secured to the pericardium 3030 or the epicardium; in another embodiment, the insert can be left to “float freely” between the pericardium and the epicardium. Of course, the insert 3050 will not float but will be held against the epicardium 3020 by forces F. In an embodiment where the insert is two-piece and self-assembles (e.g. by magnets) the second portion of the insert is is placed into the pericardium after the first portion. With the two components of the insert in the pericardial space, the magnets allow them to forcibly connect with one another.

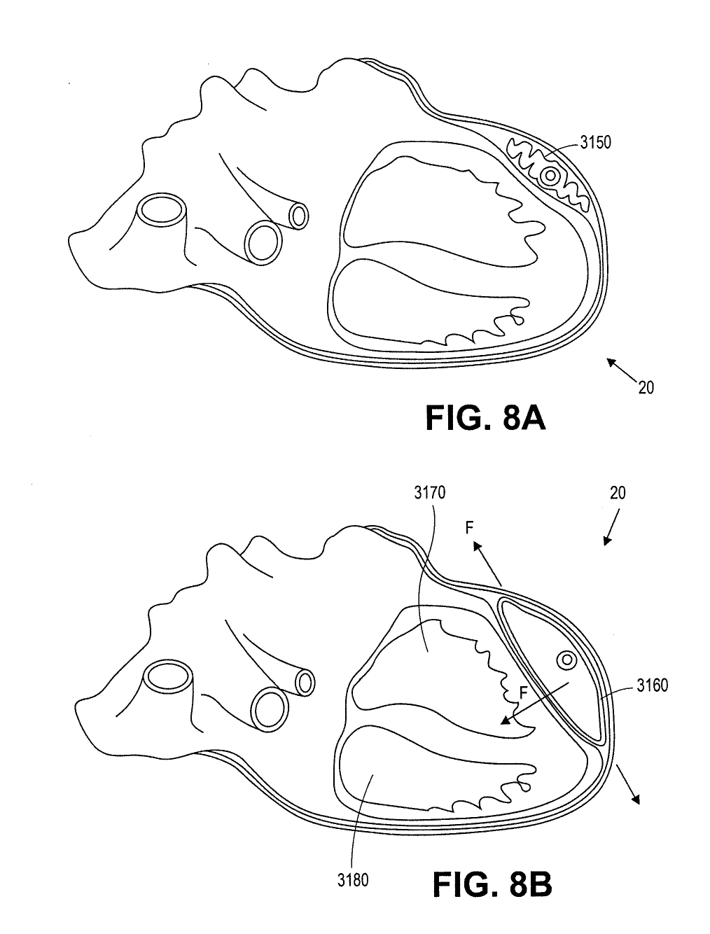

FIG. 8a depicts a cross-sectional view of the heart 20 with insert 3150 in the undeployed configuration. FIG. 8b depicts the insert in its deployed state 3160. The deployment occurs by filling the insert 3150 with fluid as described above. In some embodiments, the insert is deployed by pulling back a sheath over the insert, then subsequently filling the insert with fluid. As shown in FIG. 8b, when the insert is fully expanded, the pericardium applies force F to the insert and subsequently to the left ventricular chamber 3170. As described below and revealed in Table 1, insert 3160 can apply a force to the left ventricular chamber and the right ventricular chamber 3180 will not see the same force F′. A differential pressure can therefore be applied to the left ventricular chamber than to the right ventricular chamber.

Subsequent to placement of the insert, the tie line or access port to the insert is run through the hole in the pericardium (pericardotomy) and connected to a subcutaneous access port 1300 (FIG. 9). The access port 1300 allows for fluid administration or removal from the insert. A separate system or structure 1310 is optionally included and in some embodiments is integral to the port. This system or structure 1310 can be used for sensing or application of electrical current to the heart for pacing, defibrillation, rhythm monitoring, etc.

In some embodiments, the insert floats freely but one or more fluid line(s) (160, 180 in FIG. 1) are attached to the pericardium. The fluid lines can be rigid or have a rigid component so that the attachment to the pericardium allows maintenance of the position of the pericardial insert inside the pericardial space.

Other Methods to Treat Heart Failure with Pericardial Inserts

In other embodiments, the access port to the insert is utilized for other treatments. Because the port is accessible over time, the support structure and therefore the myocardial wall can be accessed over time as well through the port. The port can comprise a power supply or a sensor and can further comprise intelligence through a microprocessor.

In other embodiments, the inserts are used to apply other types of energy to the myocardium. For example, radiofrequency energy is applied subcutaneously and through the insert to affect the myocardium.

In another embodiment, light energy is applied to the myocardium through the insert or through the fluid of the insert. For example, a fiber optic can be placed through the support and into the insert to apply light therapy to the myocardium. Light therapy can include visible, ultraviolet, and/or infrared light therapy or combinations thereof. The light can activate or deactivate materials associated with the support structure.

In another embodiment, heat energy is applied through the inserts to treat the myocardium.

In another embodiment, heat energy is removed from the insert to cool the myocardium.

In another embodiment, electromagnetic energy is applied to the pericardium through the insert.

Experimental Verification

To verify the physiologic principles above, a series of experiments was performed. A flexible and expandable polyurethane balloon was inserted into the pericardium in a porcine animal model. A pressure measuring catheter was inserted into both the left and right ventricles. The motion of the heart walls was followed with echocardiography. The balloon was inserted over the region of the left ventricle and sequentially filled with 10 cc, 20 cc, 30 cc, 40 cc, 50 cc saline up to 160 cc. The pericardium at these filling volumes was stretched and the balloon was compressed against the left ventricle. As saline was introduced into the expandable balloon, the left ventricle became progressively compressed so that it is prevented from completely filling. At the same time, the right ventricle continued to fill normally. See Table 1 below for detailed data. Pressure data in Table 1 is expressed as systole/diastole (mean over time), with all pressures provided in mm Hg.

TABLE 1

| | | | | | | | | | | | balloon |
|---|---|---|---|---|---|---|---|---|---|---|---|
| Volume | baseline | 25 cc | 35 cc | 45 cc | 60 cc | 80 cc | 90 cc | 120 cc | 140 cc | 160 cc | out |
| Left | 80/7 (40) | 70/13 | | 75/12 | 78/13 | 69/13 (35) | 75/20 (43) | 68/20 (44) | 62/11 (36) | 57/14 | 80/15 (45) |
| Ventricle | | (35) | | (37) | (41) | | | | | (34) | |
| Pressure | | | | | | | | | | | |
| Right | 25/8 (15) | 27/10 | | 27/11 | 27/12 | | 25/10 (17) | 26/10 (28) | 26/12 (18) | 26/14 | 28/12 (20) |
| Ventricular | | (18) | | (17) | (19) | | | | | (20) | |
| Pressure | | | | | | | | | | | |
| Balloon | NA | 12/8 | 15/10 | | 15/10 | 22/15 (18) | 35/20 (25) | 25/15 (18) | 40/25 (28) | 26 | 0 |
| Pressure | | (10) | (13) | | (12) | | | | | | |
| Cardiac | | 4.4 | | | 6.2 | | 5.9 | 4.6 | 4.5 | 4.7 | 8 |
| Output | | | | | | | | | | | |
| Heart Rate | 90 | 60 | | 63 | 65 | | 67 | 71 | 73 | 80 | 70 |
| Echo EF | 55% | 50% | | | 55% | | 55% | | | 35% | 55% |
| SVO2 | 72% | 55 | | | 63% | | 65% | 63% | 65% | 57% | 79% |
| Wedge | 13 | 17 | | | 19/15 | | 20/10 (15) | 25/17 (19) | | 21/18 | 15/13 (14) |
| | | | | | (16) | | | | | (20) | |
| Pulmonary | 20/10 | | | | | | | | | | |
| artery | | | | | | | | | | | |
| pressure | | | | | | | | | | | |
| Comments | | | | | | | | | | Low | EKG |
| | | | | | | | | | | voltage | normalized |
| | | | | | | | | | | EKG | |
| | | | | | | | | | | reading | |

After the 160 cc volume caused almost complete collapse of the left ventricle, the balloon was removed from the pericardium. The left ventricle immediately returned to its pre-balloon form in which the left ventricle vigorously contracted. As can be seen in Table 1, the ejection fraction (measure of the finctioning of the heart) increased to its pre-balloon levels. Similarly, the mixed venous oxygen saturation (measure of cardiac output) returned to its pre-balloon levels. The pressure inside the ventricle decreased as the balloon was filled and similarly returned to its baseline state after the balloon was removed. The maximum pressure inside the pericardial balloon was 28 mm Hg which was high enough to cause the hemodynamic compromise seen in Table 1. Therefore, in this experiment, the useful range of pressure inside the insert is below 28 mm Hg and above zero.

Follow up of the effect of this support structure on the heart revealed that further instillation or removal of fluid could alter the cardiac hemodynamics. These chronic data show that regions of the wall of the heart can be selectively treated while not treating other regions of the heart and that this ability continues over time after the implant.

Further follow up reveals that the initial pressure created in the support structure holds the support structure in place while the support structure heals into place.

Introduction of Fluid Directly into the Potential Pericardial Space

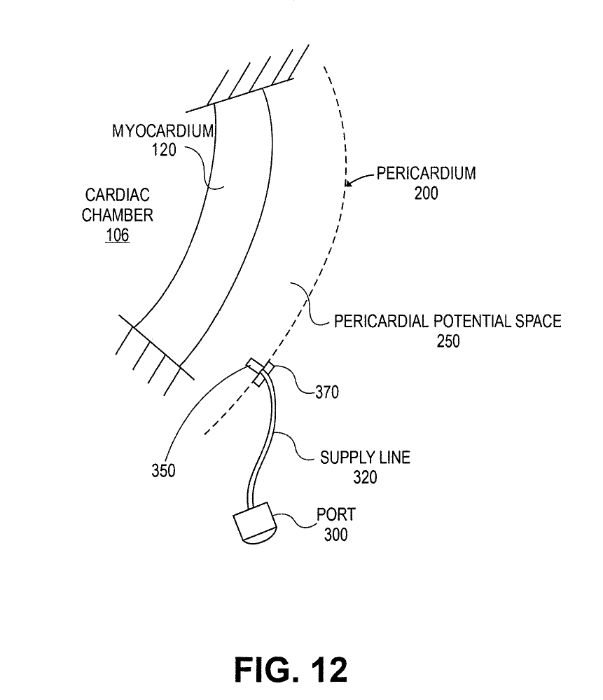

FIG. 12 depicts a section of the heart. Cardiac chamber 100 is the inner region of the heart where blood enters and then is pumped out. The pericardial potential space 250 can be filled with fluid and is wholly contained in the sense that it can be filled with fluid under pressure. The outer region of the pericardial space is the pericardium 200. A fluid delivery nozzle 350 allows for communication between a port 300 and the nozzle 350. The nozzle allows fluid to be pushed into the pericardial space 250. Seal 370 ensures that the fluid cannot escape the pericardial potential space 250. Port 300 is designed to be placed inside the patient or outside the patient. It can be implanted in the subcutaneous region or in the abdominal or chest cavity. Fluid can be injected through the port from outside the patient to the pericardial potential space 250. The fluid can be placed under a known and controllable pressure to control expansion of the myocardium 120 and prevent the unstable situation during heart failure.

What is claimed is:

1. A method of managing a heart failure patient, the patient anatomically having skin, a costal cartilage, a xiphoid and a heart, the heart comprising a left ventricle, a right ventricle, a left atrium, a right atrium, an epicardium, a pericardium and a pericardial space between the epicardium and the pericardium, the method comprising:
placing a support structure between the epicardium and the pericardium such that a force is transmitted from the pericardium through the support structure to a selected region of the epicardium; and
leaving the support structure in place between the epicardium and the pericardium postoperatively.

2. The method of claim 1 wherein placing the support structure comprises:
placing a guide wire into the pericardial space through a puncture in the skin;
positioning the guide wire over a region of the heart intended to be supported;
delivering the support structure over the guide wire to the pericardial space; and removing the guide wire.

3. The method of claim 1 wherein placing the support structure comprises: placing a flexible sheath into the pericardial space through an opening in the skin;
positioning the sheath adjacent to a region of the heart to support; delivering the support structure through the sheath to the pericardial space; and removing the sheath.

4. The method of claim 3 wherein the sheath is placed through an incision made in close proximity to the xiphoid.

5. The method of claim 1 wherein the support structure further comprises an extrapericardial extension and wherein the extrapericardial extension further comprises at least one securing portion that secures the support structure outside the pericardium.

6. The method of claim 1 further comprising securing the support structure in place without sutures.

7. The method of claim 1 wherein the support structure is delivered through an opening in the pericardium no larger than about 1.5 cm.

8. The method of claim 1 wherein the pericardium is maintained substantially intact while placing the support structure.

9. The method of claim 1 wherein the support structure is held in place inside the pericardium by friction between the support structure and the pericardium, said friction created by instillation of fluid into the support structure.

10. The method of claim 1 wherein the support structure is expandable.

11. The method of claim 10 wherein the support structure is expandable with a fluid.

12. The method of claim 10 wherein said support structure is constructed to substantially cover one of the ventricles but not the other.

13. The method of claim 11 wherein said fluid expandable support structure is set at the time of implantation to reach a pressure of less than about 20 mm Hg when the heart is expanded during diastole.

14. The method of claim 1 wherein said support structure applies a force substantially only to the left ventricle and not to the right ventricle.

15. The method of claim 1 further comprising the step of adjusting said support structure such that said support structure transmits less than 30 mm Hg to the selected region of the epicardium through transfer of force from the pericardium through the support structure to the selected region of the epicardium.

16. The method of claim 15 wherein said selected region of the epicardium is the left ventricle.

17. The method of claim 15 wherein said selected region of the epicardium includes at least a portion of one of the atria.

18. The method of claim 2 wherein the support structure comprises a plurality of separate segments.

19. The method of claim 18 wherein the plurality of separate segments is delivered over the guide wire one segment at a time.

20. The method of claim 18 further comprising interconnecting the separate segments after they are delivered over the guide wire.

21. The method of claim 20 wherein the separate segments are interconnected by joining magnets located on the segments.

22. The method of claim 1 further comprising removing the support structure at some time post-operatively.

23. The method of claim 1 further comprising post-operative adjustment of said force transmitted between the pericardium and the epicardium by the support structure to modulate a therapeutic effect of the support structure.

24. The method of claim 1 wherein said support structure further comprises an electrical conducting portion and said electrical conducting portion is activateable after implantation to create a desired therapeutic effect.

25. A method of cardiac treatment comprising:
providing an implantable insert having an inflated state and a deflated state;
placing the insert into a patient's body in the deflated state and positioning the insert over a selected region of the patient's heart;
inflating the insert to put pressure on the selected region without applying substantial pressure to other regions of the heart; and
maintaining pressure in the insert for an extended period of time to create a desired therapeutic effect on the region.

26. The method of claim 25 wherein the desired therapeutic effect comprises inhibiting cardiac dilation of the region.

27. The method of claim 25 wherein the insert is placed between the epicardium and the pericardium.

28. The method of claim 25 flurther comprising deflating and removing the insert.

29. The method of claim 28 wherein pressure is maintained in the insert for at least 1 month before removal.

30. The method of claim 28 wherein pressure is maintained in the insert for at least 6 months before removal.

31. The method of claim 28 wherein pressure is maintained in the insert for at least 2 years before removal.

32. The method of claim 25 wherein pressure is maintained in the insert such that it does not exceed a maximum of about 5 mm Hg.

33. The method of claim 25 wherein pressure is maintained in the insert such that it does not exceed a maximum of about 25 mm Hg.

34. The method of claim 25 wherein pressure is maintained in the insert such that it does not exceed a maximum of about 40 mm Hg.

35. The method of claim 25 wherein a minimum pressure of about 5 mm Hg is maintained in the insert.

36. The method of claim 25 wherein a minimum pressure of about 10 mm Hg is maintained in the insert.

37. The method of claim 25 wherein a minimum pressure of about 20 mm Hg is maintained in the insert.

38. The method of claim 25 wherein a minimum pressure of about 5 mm Hg is maintained in the insert.

39. The method of claim 25 wherein the selected region is the left ventricle.

40. The method of claim 25 further comprising placing a reservoir port in the patient's skin and changing the pressure in the insert by adding fluid to or removing fluid from the insert through the port.

41. The method of claim 25 further comprising placing a fluid pump in the patient's body in fluid communication with the insert.

42. The method of claim 25 further comprising placing at least one sensor in the patient's body for monitoring a parameter associated with the insert.

43. The method of claim 42 wherein the parameter is pressure.

44. The method of claim 42 wherein the parameter is strain.

45. The method of claim 42 wherein the parameter is volume.
